(19) United States
(12) Patent Application Publication
(10) Pub. No.: US 2004/0191906 A1
(43) Pub. Date: Sep. 30, 2004

(54) Method for selective enhancement of cell growth

(76) Inventor: Asher Holzer, Haifa (IL)

Correspondence Address: Mark Friedman, Bill Polkinhorn, Discover Dispatch, 9003 Florin Way, Upper Marlboro, MD, 20772, US

(21) Appl. No.: 10/485,472

(22) PCT Filed:
(86) PCT No.: PCT/IL02/00632

Related U.S. Application Data
(60) Provisional application No. 60/308,813, filed on Aug. 1, 2001.

(51) Int. Cl. C12N 5/02

(52) U.S. Cl. 435/383

(57) ABSTRACT

A method for enhancement of cell growth, the method including the steps of (a) providing a system including: (i) an ultrasound transducer; (ii) an interface medium for promoting ultrasound transmission, and (iii) at least a first type of cells, disposed within a growth medium; (b) producing micro-vibrations by means of the ultrasound transducer, and (c) applying the micro-vibrations to the first group of cells, so as to promote growth of the cells, wherein the micro-vibrations have a frequency within a range of 20 kilo Hz to 4 mega Hz.

This patent application draws priority from U.S. Provisional Patent Application, Serial No. 60/303,813.

### FIELD AND BACKGROUND OF THE INVENTION

The present invention relates to a method for the selective enhancement of cell growth and, in particular, it concerns a method of triggering such a selective growth-enhancement process, and controlling the process, by application of micro-vibrations.

“Stem cells” is a term to describe precursor cells that can give rise to multiple tissue types. There are important distinctions, however, regarding how developmentally plastic these cells are; that is, how many different paths they can follow and to what portion of a functioning organism they can contribute. Totipotent stem cells am cells that can give rise to a fully functional organism as well as to every cell type of the body. Pluripotent stem cells are capable of giving rise to virtually any tissue type, but not to a functioning organism. Multipotent stem cells are more differentiated cells (that is, their possible lineages are less plastic/more determined) and thus can give rise only to a limited number of tissues. For example, a specific type of multipotent stem cell called a mesenchymal stem cell has been shown to produce bone, muscle, cartilage, fat, and other connective tissues. (See Pittenger, M. F., et al., “Multilineage Potential of Mesenchymal Stem Cells”, Science, 284: 143-147, 1999).

There are many potential sources for stem cells. Embryonic stem (ES) cells are derived from the inner cell mass of a blastocyst (a very early embryo). Embryonic germ cells are collected from fetal tissue at a somewhat later stage of development (from a region called the gonadal ridge), and the cell types that they can develop into may be slightly limited. Adult stem cells are derived from mature tissue. Even after complete maturation of an organism, cells need to be replaced (a good example is blood, but this is true for muscle and other connective tissue as well, and may be true for at least some nervous system cells). Because these give rise to a limited number of cell types, they are perhaps more accurately referred to as multipotent stem cells, as discussed above.

Much of the experimental data collected over recent years were produced in experiments using mice. There is an abundance of stem cell lines from mammals, including some from human beings. ES cells are valuable scientifically because they combine three properties not found together in other cell lines:

Replication in an essentially indefinite fashion, without undergoing senescence or mutation of the genetic material. Consequently, ES cells are a large-scale and valuable source of cells.

Genetic normality, as is evidenced by a series of genetic tests and functionally, as shown by the creation of mice with genomes derived entirely from ES cells. In mice these cells are developmentally totipotent; when inserted into an early embryo, they join the host cells to create a normal mouse, differentiating into every cell type of the body (it is this property that earns them the name “stem cell of the body”).

Differentiation capability: ES cells can differentiate into many cell types in tissue culture, including neurons, blood cells and cardiac and skeletal muscle. The normal embryo has about 100 cells with the properties of ES cells that exist for about one day and then develop into more advanced cell types.

Adult-derived stem cell therapies will complement, but cannot replace, therapies that may be eventually obtained from ES cells. They do have some advantages. For example, adult stem cells offer the opportunity to utilize small samples of adult tissues to obtain an initial culture of a patient's own cells for expansion and subsequent implantation (an autologous transplant). This process avoids any ethical or legal issues concerning sourcing, and also protects the patient from viral, bacterial, or other contamination from another individual. With proper manufacturing quality controls and testing, allogeneic adult stem cells (cells from a donor) may be practical as well. Already in clinical use are autologous and allogeneic transplants of hematopoietic stem cells that are isolated from mobilized peripheral blood or from bone marrow by positive selection with antibodies in commercial devices. In general, there is less ethical concern over their initial source. Additionally, since they normally differentiate into a narrower set of cell types, directing them to a desired fate is more straightforward. However, many cells of medical interest cannot, as of yet, be obtained from adult-derived cell types. Production of large numbers of these cells is even more difficult than is the case for ES cells.

ES cell technology may well be transformative in opening scientific arenas that to date have been closed.

The economic and psychological tolls of chronic, degenerative, and acute diseases in the United States are enormous. It has been estimated that up to 128 million people suffer from such diseases; thus, virtually every citizen is effected directly or indirectly. The total costs of treating diabetes, for example, is approaching $100 billion in the United States alone. As more research takes place, the developmental potential of different kinds of stem cells will become better understood. As the science is understood now, adult stem cells are limited in their potential to differentiate. Embryonic germ cells have a great differentiation capacity, and embryonic stem cells are thought to be able to differentiate into almost any tissue. Thus, different types of stem cells could have different applications.

Enhancing the growth of stem cells, while inhibiting or depressing the growth of others, appears to be a key factor for making this technology feasible. However, triggering neural stem cells to differentiate into the various kinds of neurons that make up the human brain or other organ, is most complicated.

There is therefore a recognized need for, and it would be highly advantageous to have, a method and system for enhancing the growth of a specific type of cell, while inhibiting or depressing the growth of other types. It would be of further advantage for such a method and system to be simple, inexpensive, chemical-free, and environmentally-friendly.

### SUMMARY OF THE INVENTION

The present invention relates to a method of triggering a selective growth-enhancement process in living cells, and controlling the process, by application of micro-vibrations.

According to the teachings of the present invention there is provided a method for enhancement of cell growth, the method including the steps of: (a) providing a system including (i) an ultrasound transducer; (ii) an interface medium for promoting ultrasound transmission, and (iii) at least a first type of cells, disposed within a growth medium; (b) producing micro-vibrations by means of the ultrasound transducer, and (c) applying the micro-vibrations to the first group of cells, so as to promote growth of the cells, wherein the micro-vibrations have a frequency within a range of 20 kilo Hz to 4 mega Hz.

According to further features in the described preferred embodiments, the method further includes the step of: (d) immersing the first type of cells, at least partially, in the interface medium.

According to still further features in the described preferred embodiments, the method further includes the step of: (d) completely immersing the first type of cells in the interface medium.

According to still further features in the described preferred embodiments, the micro-vibrations have a frequency within a range of 20 kilo Hz to 0.5 mega Hz.

According to still further features in the described preferred embodiments, the micro-vibrations have an amplitude within a range of 0.1 microns to 200 microns.

According to still further features in the described preferred embodiments, the micro-vibrations have an amplitude within a range of 10 microns to 200 microns.

According to still further features in the described preferred embodiments, the micro-vibrations have a total power density of up to 10 watts per cubic centimeter.

According to still further features in the described preferred embodiments, the system further includes: (iv) at least a second type of cells, and step (c) includes applying the micro-vibrations to both the first type of cells and the second type of cells, so as to induce selective growth of the first type of cells with respect to the second type of cells.

According to still further features in the described preferred embodiments, the method further includes the step of: (d) immersing the first type of cells and the second type of cells, at least partially, in the interface medium.

According to still further features in the described preferred embodiments, the micro-vibrations are applied in-vivo.

According to still further features in the described preferred embodiments, the micro-vibrations are applied ex-vivo.

According to still further features in the described preferred embodiments, the system further includes: (iv) at least a second type of cells, and step (c) includes applying the micro-vibrations to both the first type of cells and the second type of cells, so as to enhance growth of the first type of cells while simultaneously depressing growth of the second type of cells.

According to still further features in the described preferred embodiments, the micro-vibrations are applied for periods within a range of milliseconds to days.

According to still further features in the described preferred embodiments, the micro-vibrations are applied so as to enhance growth of stem cells within said first type of cells.

According to still further features in the described preferred embodiments, the micro-vibrations are applied to a stent located in proximity to a neuron band, so as to enhance growth of nerve tissue.

According to still further features in the described preferred embodiments, the ultrasound transducer has a tip made of titanium.

According to still further features in the described preferred embodiments, the micro-vibrations are applied to a coronary stent, so as to enhance growth of myocardium tissue.

According to still further features in the described preferred embodiments, the micro-vibrations are applied to a coronary stent, so as to enhance revascularization.

According to still further features in the described preferred embodiments, the micro-vibrations are applied to a coronary stent, so as to inhibit restenosis.

According to still further features in the described preferred embodiments, at least one pellet is pre-disposed within said growth medium, so as to enhance growth of said first type of cells.

According to still further features in the described preferred embodiments, the pellet is made of titanium.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic cross-sectional view of an ultra-sonic micro-vibration producing system of the present invention;

FIGS. 2a-b are schematic cross-sectional views of a preferred embodiment, in which ultra-sonic transducers are attached to the vessel containing the seeds or culture, with (FIG. 2a) and without (FIG. 2b) a support ring for a grid;

FIG. 3 is a schematic diagram of a typical sonicator system for use in conjunction with the present invention, and

FIG. 4 is a schematic diagram of a preferred embodiment in which pellets, preferably made of titanium, are disposed within the growth medium (in-vivo or ex-vivo).

### DESCRIPTION OF THE PREFERRED EMBODIMENTS

The present invention relates to a method of triggering a selective growth-enhancement process in living cells, and controlling the process, by application of micro-vibrations.

The principles and operation of the cell growth-enhancement process according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

The growth enhancement and differentiation of cell growth can be performed ex-vivo (e.g., in a petri dish), and in vivo in humans, fetuses, animals, plants, and others, using micro-vibrations applied on the sample target. Such micro-vibrations can be applied using ultrasonic transducers at various frequencies.

The inventive method enhances and accelerates the growth of a particular type of cells, and increases the growth affinity, i.e., the growth of one type of cells relative to another cell type located in the same proximity. This is also termed “selective growth enhancement”. The method described can selectively increase the growth of one type of cell over other types. Although the exact mechanism of such a process is not yet fully understood, there are clear indications, supported by various experiments of accelerated growth of certain types of cells, bacteria, or parts of plants or organs relative to other cells, parts of plants, and organs, as well as over a control group tested under identical conditions (but not exposed to ultra sonic micro-vibrations).

The method and apparatus described herein can be used to simultaneously inhibit the growth of other cells. It is not clear at this stage if the inhibition is due to the direct application of the method and the apparatus on the tested object, or due a deficiency in materials and other resources, due to the preferred accelerated growth of other cells that compete for the same resources, and growth factors. Another possible explanation is that the instant invention stimulates the activity or the production of some enzymes, while inhibiting or slowing down the production, activity, etc., of some other enzymes. As a non-limiting example, stem cells can be better grown in petri dishes with amino acids and growth factors using the method and apparatus of the instant invention.

Another example is the accelerated growth of plant seeds exposed to micro-vibrations directed towards the media of the plant seeds.

Another example is the use of such micro-vibrations to enhance the healing process of wounded tissues or organs, and to enhance the growth of human (and non-human) nerves while applying micro-vibrations at various frequencies, various amplitudes and of varying duration to the proximity of the wounded nerve. The method can be used to enhance the connections, and the healing, of a nerve that has been severed, by applying this method to the proximity of the wounded nerve, and/or to the scaffold stent attached to it, and/or to the gel or other ‘bandage’ surrounding the wounded nerve. For example, spine injury patients suffering from a damaged spinal nerve system may be able to benefit from the application of such micro-vibrations, and ultra-sonic energy, to the wounded area, with or without the presence of other drugs, stem cell islets, or other stimulating growth factors.

Another example is the use of such a method for the acceleration of bone healing, by applying such micro-vibrations to the proximity of the wounded bone with or without the presence of an external biomedical agent (e.g., gel/ointment with certain drugs, stem cell islets. etc.).

Another non-limiting example is the use of this novel technique in conjunction with revascularization of blood vessels, (for example in the myocardium) avoiding and treating restenosis of pre-treated blood vessels, (with stents), in conjunction with cancer treatment, and age-related disorders, or even as part of an anti-aging treatment.

In particular, the present invention is a method of selective enhancement of cell growth for a particular type of cell, as well as enabling this type of cells to grow faster and better than other types of cells disposed proximately and/or similarly treated. It can trigger the growth of certain cells, while inhibiting the growth of others. Thus, the contribution of the present invention can be utilized in the area of stem cells growth and in cultivation of ex-vivo of all kind of cells in particular, stem cells, or other cells that exist in small percentage in a matrix. At the same time, it can be used in-vivo, for enhancing or inhibiting the growth of certain cells, trigger the production of certain enzymes, glands, and other metabolic processes within the body.

The present invention can be also utilized in the potential inhibition of cancer cells growth, over “good” benign cells.

The present invention can help in the promotion of nerve rejuvenation, and growth, where it is known that standard existing techniques can not effectively cause the nerves to grow, or to be rejuvenated, nor to be linked back to another nerve in the immediate vicinity.

The present invention can be utilized to promote growth of whole organs, either in plants, animals, or in humans, such that one can grow, by way of example, plants with more, larger and better quality seeds, fruits, or leaves.

Animals such as cows can be manipulated to produce more milk by accelerating the growth of certain organs, glands, or by stimulation of enzyme production.

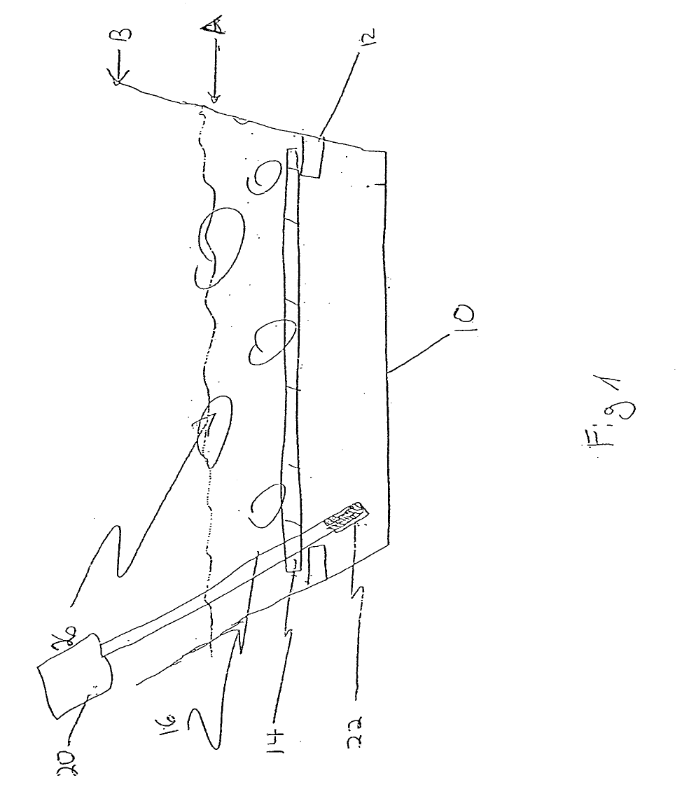

Referring now to the drawings, FIG. 1 describes the experimental setup and the apparatus, including sonicator positions and locations.

In a standard petri dish 10, a support ring 12 supports a metal grid or net 14, preferably made of titanium. A piece of cotton 16 is located above grid 14.

Water is added to fill petri dish 10 to level A, such that the whole piece of cotton 16 is soaked in water, or to level B, such that at least partial immersion is achieved. This is important because a portion of the micro-vibrational energy is transmitted via the water. A sonicator 20 with a horn and tip 22 made of titanium is disposed and immersed in the water. Horn and tip 22 are designed with specific geometries enabling them to be tuned to the frequencies desired. It must be emphasized that various commercially-available products are suitable for producing the requisite ultra-sonic micro-vibrations, including devices produced by Sonics and Material (Connecticut), Misonics (Long Island, N.Y.) and Branson (Germany).

Preferably, sonicator 20 is suspended, primarily to avoid vibration of petri dish 10, and at least partially immersed in water.

In another configuration, support ring 12, in addition to providing support for grid 14, also acts as the vibrating element.

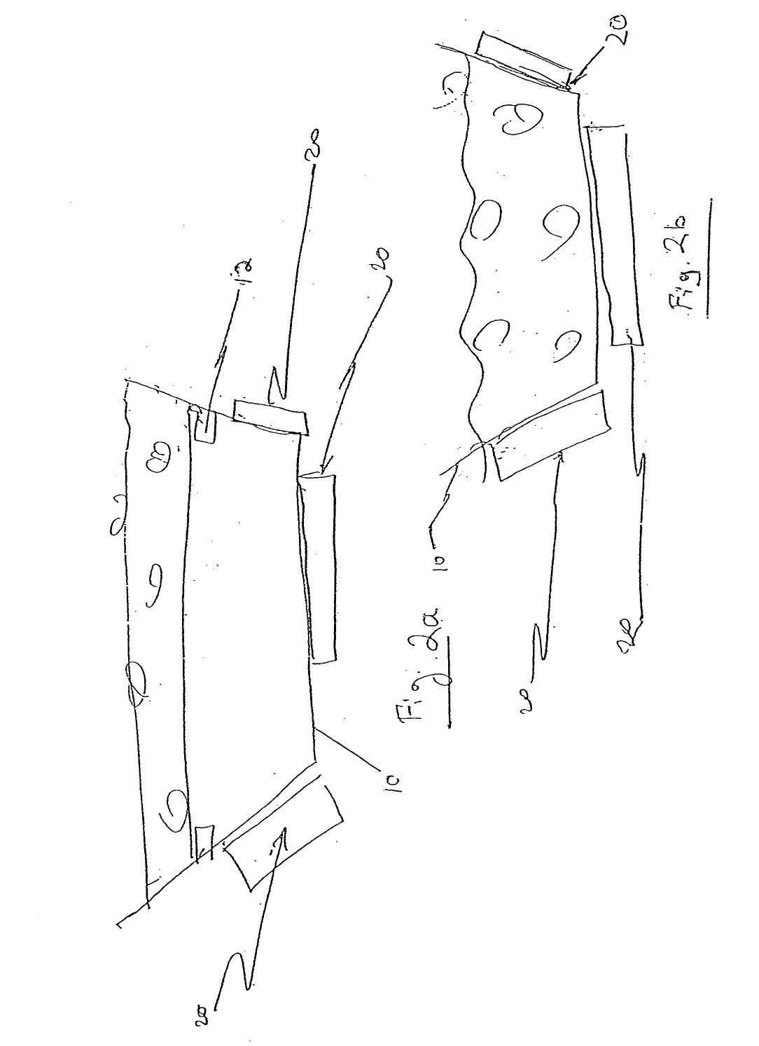

In another preferred embodiment, illustrated in FIGS. 2a-b, petri dish 10 is made of titanium. At least one ultra-sonic transducer 20 is attached to petri dish 10, with (as shown in FIG. 2a) or without (as shown in FIG. 2b) support ring 12.

The ultrasound transducers can be arranged in a geometry, so as to focus their transmitted energy, or in a ‘phased array’ mode, so as to focus the energy both in geometry and in time.

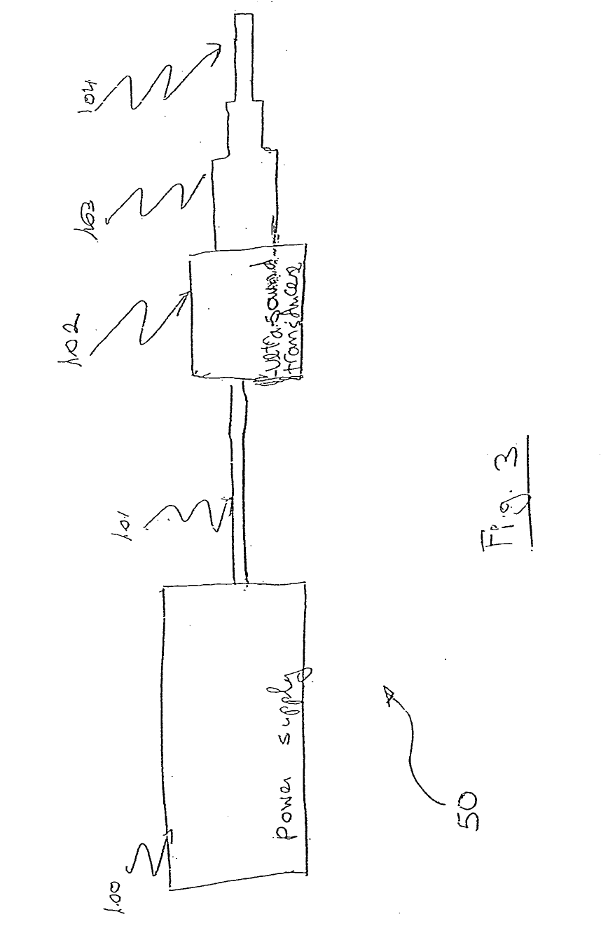

FIG. 3 is a schematic diagram of a typical sonicator system for use in conjunction with the present invention. Sonicator system 50 contains a power supply 100 for producing the requisite energy at the desired frequencies, a coaxial cable 101 that transmits this energy to an ultrasound transducer 102 located in a holding block box. The horn 103 and tip 104 of ultrasound transducer 102 are physically attached to the transducer at one end thereof. Horn 103 and tip 104 are preferably made of titanium. The geometries of horn 103 and tip 104 are tuned to the desired frequency or frequencies, as is known to those skilled in the art.

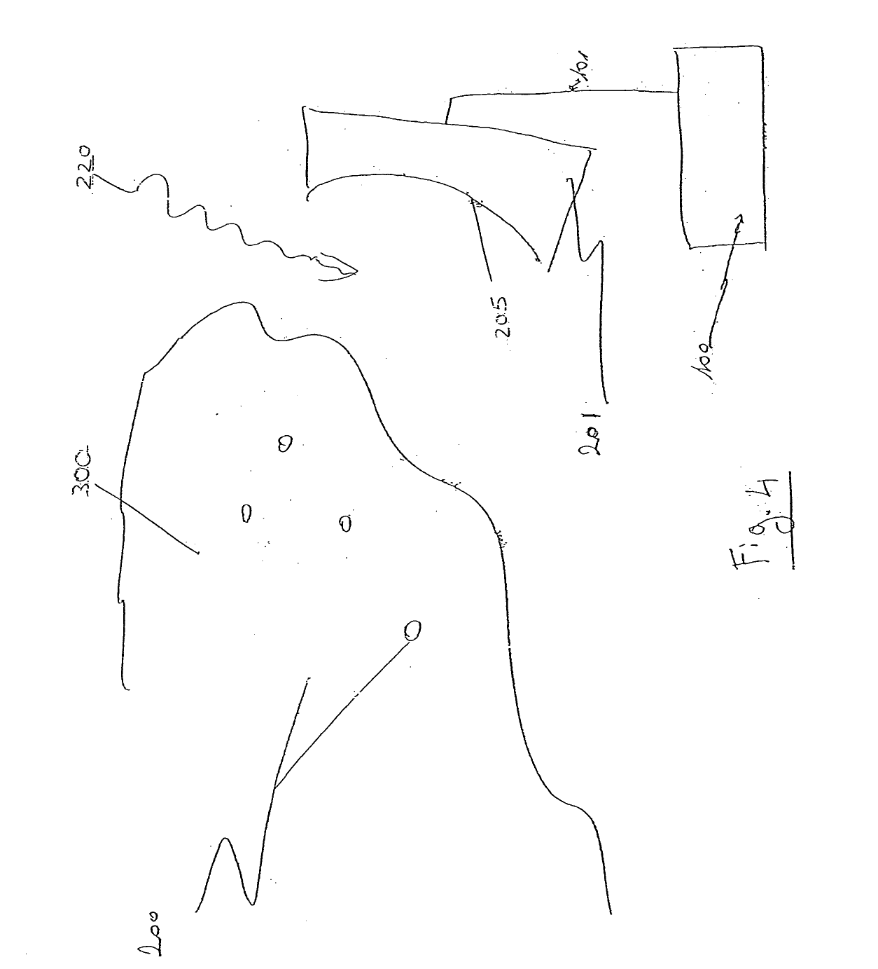

FIG. 4 is a schematic diagram of a preferred embodiment in which beads or pellets 200, preferably made of titanium, are disposed within growth medium 300. Growth medium 300 may be in-vivo. e.g., within a human/animal body or a plant, or ex-vivo, e.g., a petri dish. At least one ultra-sound transducer 201 is disposed so as to enable a focused beam to be directly applied to the target zone within growth medium 300. Preferably, pellets 200 are be pre-disposed within growth medium 300. An interface layer 220, which is preferably water, a gel, etc., is preferably used to enhance the transmission of micro-vibrations from transducer 201 to growth medium 300.

The transmission surface 205 of transducer 201 can be flat or curved, with or without a phased array option.

### EXAMPLES

Reference is now made to the following examples, which together with the above description, illustrate the invention in a non-limiting fashion.

### Example 1

In several petri dishes, bean seeds were inserted in cotton soaked in tap water. An ultra sound actuator, called a sonicator, which produces ultra-sonic micro-vibrations at 20 kilo hertz (with a tuned titanium tip), applied micro-vibrations to the proximity of the bean seeds. The power emitted from the tip was under 1 watt.

The amplitude of the vibrating tip was in the range of several microns to 50 microns.

A second set of petri dishes was disposed next to the tested set, and was exposed to identical conditions (e.g., water, light, humidity, etc.), as a control set for comparative purposes.

The length of the roots and the length of the leaves were monitored daily for each bean plant, and averages were compiled for the test set and for the control set. At the end of the experiment, the roots and leaves were cut and weighed, and the results were recorded.

It was found, surprisingly, that under certain frequencies, amplitudes and duration of applying the micro-vibrations, the growth of the roots was 2.8 times faster when applying the ultra-sound energy (compared to a control group, under the same conditions, but without the presence of the micro-vibrations). In another experiment in which a different combination of frequencies and amplitudes was applied, the rate of growth of the leaves was 1.9 times the rate of growth of the control group.

Looking at typical results provided in Table 1, it is easy to deduce that when the system is tuned (frequencies, location of micro-vibrations, etc.) to accelerate the growth of roots, the leaves growth is retarded, and vice versa. It is not clear at this stage if the delayed growth is due to a direct influence of the micro-vibrations, or due to the fact that there may be a temporary lack of resources to the plant due to the accelerated growth of the roots. No matter what the explanation is, the practical end results are that the growth of one part of the plant can be accelerated, while the growth other parts can be inhibited. This provides a strong indication that a preferred, selective growth of one organ/type of cells, can be achieved.

Similarly, the effect of ultra-sonic micro-vibrations on the growth of pea seeds was investigated.

TABLE 1
COMBINED INTEGRATIVE RESULTS OF SEVERAL
EXPERIMENTS OF BOTH LEAVES AND ROOTS
(The results are in weight percentages relative to
the control group that is 100 percent)

| | |
|---|---|
| TEST GROUP | CONTROL GROUP |

| | leaves | roots | leaves | roots |
|---|---|---|---|---|
| Set up 1 | 68 | 280 | 100 | 100 |
| Set up 2 | 190 | 86 | 100 | 100 |

These type of experiments were performed several times under different environmental conditions of light, amount of water, fertilizers, and temperature, and all achieved similar results: relative and selective growth of the roots and the leaves can be controlled by applying ultrasound micro-vibrations to their proximity. Qualitatively-similar results were obtained in experiments using bean plants.

### Example 2

In another experiment, the sonicators were removed after 10 days from the test group of petri dishes and were subsequently applied to the control group samples. It was observed that after the first 10 days, the increased growth in the petri dishes that were exposed to the micro-vibrations was readily apparent, and after the ultrasound source was removed to the control group of petri dishes, the growth of the bean plants in the control group was accelerated, such that within 9 days, the growth in the two sets was substantially identical.

These results clearly show that using the method described, under the process indicated, with the apparatus described, one can manipulate the growth of these seeds, so as to selectively enhance either root growth, or leaf growth.

### Example 3

Similar experiments were conducted applying such ultra-sonic micro-vibrations in a petri dish with various cultures and yeasts. It was shown that the growth of these cultures, and some of the ingredients produced by the yeast and the bacteria can be controlled (the amount produced, as well as their presence, in some cases).

The micro-vibration method described hereinabove can be applied on certain areas of human and animal bodies either externally, percutaneously, via an internal source, or using seeds located in the body that are subjected to external micro-vibrations, for the purpose of enhanced curing of disease, injuries, selective enhancement of growth of a particular type of cell, or selective inhibition of another type of cell. The micro-vibration method disclosed herein can be applied along with various known treatments.

For example, the method can be applied to a stent located around the spin, or at the proximity of a neuron band, for the purpose of enhancing the growth or rejuvenation of nerves. The method can be used in conjunction with other therapeutic modalities such as stem cells, growth factors, and various drugs.

The method may also be applied to a coronary stent, for the purpose of enhancing revascularizations, avoiding restenosis (i.e., a re-narrowing or blockage of an artery at the same site where treatment, such as an angioplasty or stent procedure, has already taken place), and the re-growth of myocardium tissue.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for enhancement of cell growth, the method comprising the steps of:
(a) providing a system including:
(i) an ultrasound transducer;
(ii) an interface medium for promoting ultrasound transmission, and
(iii) at least a first type of cells, disposed within a growth medium;
(b) producing micro-vibrations by means of said ultrasound transducer, and
(c) applying said micro-vibrations to said first group of cells, so as to promote growth of said cells,
wherein said micro-vibrations have a frequency within a range of 20 kilo Hz to 4 mega Hz.

2. The method of claim 1, further comprising the step of:
(d) immersing said first type of cells, at least partially, in said interface medium.

3. The method of claim 1, further comprising the step of:
(d) completely immersing said first type of cells in said interface medium.

4. The method of claim 1, wherein said micro-vibrations have a frequency within a range of 20 kilo Hz to 0.5 mega Hz.

5. The method of claim 4, wherein said micro-vibrations have an amplitude within a range of 0.1 microns to 200 microns.

6. The method of claim 1, wherein said micro-vibrations have an amplitude within a range of 10 microns to 200 microns.

7. The method of claim 2, wherein said micro-vibrations have an amplitude within a range of 10 microns to 200 microns.

8. The method of claim 1, wherein said micro-vibrations have a total power density of up to 10 watts per cubic centimeter.

9. The method of claim 1, wherein said system further includes:
(iv) at least a second type of cells,
and wherein step (c) includes applying said micro-vibrations to both said first type of cells and said second type of cells, so as to induce selective growth of said first type of cells with respect to said second type of cells.

10. The method of claim 9 further comprising the step of:
(d) immersing said first type of cells and said second type of cells, at least partially, in said interface medium.

11. The method of claim 9, wherein said micro-vibrations are applied in-vivo.

12. The method of claim 9, wherein said micro-vibrations are applied ex-vivo.

13. The method of claim 10, wherein said micro-vibrations have an amplitude within a range of 0.1 microns to 200 microns.

14. The method of claim 1, wherein said system further includes:
(iv) at least a second type of cells, and wherein step (c) includes applying said micro-vibrations to both said first type of cells and said second type of cells, so as to enhance growth of said first type of cells while simultaneously depressing growth of said second type of cells.

15. The method of claim 14, further comprising the step of:
(d) immersing said first type of cells and said second type of cells, at least partially, in said interface medium.

16. The method of claim 14, wherein said micro-vibrations are applied in-vivo.

17. The method of claim 14, wherein said micro-vibrations are applied ex-vivo.

18. The method of claim 15, wherein said micro-vibrations have an amplitude within a range of 0.1 microns to 200 microns.

19. The method of claim 1, wherein said micro-vibrations are applied for periods within a range of milliseconds to days.

20. The method of claim 14, wherein said micro-vibrations are applied so as to enhance growth of stem cells within said first type of cells.

21. The method of claim 14, wherein said micro-vibrations are applied to a stent located in proximity to a neuron band, so as to enhance growth of nerve tissue.

22. The method of claim 14, wherein said ultrasound transducer has a tip made of titanium.

23. The method of claim 14, wherein said micro-vibrations are applied to a coronary stent, so as to enhance growth of myocardium tissue.

24. The method of claim 14, wherein said micro-vibrations are applied to a coronary stent, so as to enhance revascularization.

25. The method of claim 14, wherein said micro-vibrations are applied to a coronary stent, so as to inhibit restenosis.

26. The method of claim 1, wherein at least one pellet is pre-disposed within said growth medium, so as to enhance growth of said first type of cells.

27. The method of claim 26, wherein said at least one pellet is made of titanium.
